(19) United States
(12) Patent Application Publication
(10) Pub. No.: US 2009/0227924 A1
(43) Pub. Date: Sep. 10, 2009

(54) PIXILATED BANDAGE

(75) Inventors: Robert A. Conrad, Spring, TX (US); Charles Conrad, Spring, TX (US); Mike Garretson, Houston, TX (US); Walter V. Klemp, Houston, TX (US); Scott Thielman, Seattle, WA (US)

Correspondence Address: FULBRIGHT & JAWORSKI L.L.P, 2200 ROSS AVENUE, SUITE 2800, DALLAS, TX, 75201-2784, US

(73) Assignee: Zeno Corporation, Houston, TX (US)

(21) Appl. No.: 12/397,419

(22) Filed: Mar. 4, 2009

Related U.S. Application Data
(60) Provisional application No. 61/033,690, filed on Mar. 4, 2008.

(51) Int. Cl. A61F 7/08; A61F 13/00

(52) U.S. Cl. 602/2

(57) ABSTRACT

A bandage is described which includes a plurality of individual heating elements, each element being able to be individually heated to direct thermal energy to a specific area of the bandage, and a controller operable to control the plurality of individual bandages.

### CROSS-REFERENCE TO RELATED APPLICATION

The present patent application claims priority under 35 U.S.C. § 119(e) to U.S. Provisional Patent Application Ser. No. 61/033,690, filed Mar. 4, 2008, the entirety of which is herein incorporated by reference.

### TECHNICAL FIELD

The present invention relates to methods and devices for treatment of wounds. More specifically, the present invention relates to a heated bandage that enables the control of bacterial infection in a wound and more particularly allows for sizing for a particular wound, directing varying amounts of thermal energy to a specific site on the wound and following pre-programmed routines for ongoing therapy.

### BACKGROUND OF THE INVENTION

Skin infections and irritations pose significant health and cosmetic problems. Bacterial and fungal skin infections can result in wounds on the skin or can occur as secondary infections in existing wounds. Heat has been used as an effective treatment for many types of infections such as bacterial, viral, or as a result of irritants or allergens. Heat also has other benefits when applied to a wound, including increased blood flow to the wound and increased immune system functions.

Previous thermal bandages, such as those described by U.S. Pat. No. 6,423,018 to Augustine or U.S. Pat. No. 4,962,761 to Golden, while applying heat to a wound, only apply enough thermal energy to increase blood flow, but do not provide enough thermal energy in the correct temperature range to destroy the infectious agents or inhibit the spread of infectious agents. Augustine describes a treatment temperature of 38° C. which is ineffective for treating infectious agents directly.

Further, prior art bandages are all complete devices with a single heating element for the entire device. This results in two shortcomings. First, to treat wounds of different sizes, multiple sizes of bandages must be manufactured. Second, the bandage is heated as a single unit. Since the bandages are pre-made and of uniform sizes, the bandage may not cover the wound exactly resulting in the thermal energy being applied to healthy tissue which may not be desirable. Further, heat is applied to the wound uniformly where there may be distinct advantages to applying the thermal energy non-uniformly across the wound, or only applying the thermal energy to distinct areas of the wound.

What is needed is a bandage that can be sized to a particular wound and deliver a therapeutic amount of heat to all or targeted portions of the wound. Such heat can be delivered by maintaining the wound at a constant temperature or by applying heat as a part of a periodic treatment protocol or routine.

### BRIEF SUMMARY OF THE INVENTION

A bandage is described that includes a plurality of individual heating elements, at least a subset of elements of the plurality of individual elements being able to be separately controlled independently of the other elements to direct thermal energy to a specific area of the bandage, and a controller operable to control the plurality of heating elements forming the bandage.

In another embodiment, a method of delivering thermal energy to a wound is described. The method includes placing a bandage on the wound the bandage formed of a plurality of independently controllable elements, each element including a heating element and a feedback mechanism, heating one or more of the plurality of independently controllable elements to a temperature sufficient to destroy infectious agents, and controlling the one or more of the plurality of independently controllable heating elements using the feedback mechanism to deliver a therapeutic amount of thermal energy according to a preprogrammed treatment regimen.

In yet another embodiment a bandage element is described that includes a heating element formed on a flexible circuit board and a feedback mechanism formed on the flexible circuit board operable to provide a signal indicative of the temperature of the heating element. The bandage element further includes leads extending from the heating element and feedback mechanism providing electrical connection of the heating element and the feedback mechanism to a controller wherein the bandage element is configurable with other bandage elements into an array of individually controllable bandage elements.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a diagram of an embodiment of a heated bandage according to the concepts described herein;

FIG. 2 is a diagram of an embodiment of a bandage having linear heating elements in accordance with the concepts described herein;

FIG. 3 is a diagram of a bandage having an array of heating elements in accordance with the concepts described herein;

FIG. 4 shows a simplified block diagram of the major electrical components treatment device using the concepts described herein;

FIG. 5 is a diagram illustrating the control functionality of an embodiment of the firmware for embodiments of the treatment devices described herein;

FIGS. 6A-C show a state diagram illustrating the operation of a treatment device according to the concepts described herein;

FIG. 7 is a perspective view of an embodiment of a treatment device for use with embodiments of a heated bandage as described herein; and

FIG. 8 shows a graph of the Thermal Aspect Ratio in accordance with an embodiment of the present invention.

### DETAILED DESCRIPTION OF THE INVENTION

The present invention describes methods and devices for the treatment of wounds to treat or prevent infections that can occur. The treatment involves the application of a controlled dose of thermal energy to the infected or affected tissue and thereby preventing bacteria or other infectious agents from forming in the wound, or killing bacteria or other infectious agents present in the wound, thereby speeding the recovery process. An infectious agent is any infected or irritated tissue caused by bacterial, fungal or viral infections, or other type of irritant, which can be treated through the application of a regulated amount of heat, where “treating” an infection means to slow, halt or even reverse the development of the infection and to reduce the healing time.

Infections caused by bacterial, fungal and viral infections can be effectively treated by the application of controlled quantities of heat either by the stimulation of a “heat-shock” response in the microorganism resulting in its death, impairment, dormancy or other loss of viability of the infectious agent.

The methods and devices of the present invention provide the application to an infected area of an amount of heat (thermal energy) wherein the heat is applied over one or more treatment periods in an amount sufficient to result in improved recovery times for the treated infection. An effective therapeutic amount is therefore any application or applications of heat that are capable of measurably decreasing average recovery times for a given type of infection.

Embodiments of a pixilated bandage according to the concepts described herein will be a flexible bandage element constructed from biocompatible flexible materials. Some portion of the bandage will carry an adhesive layer for affixing the bandage to the skin. In preferred embodiments, the remaining portion(s) of the bandage will contain a number of heating zones. While it is preferred that the heating zone not overlap with the adhesive portions of the bandage, depending on materials and the thermal properties of the adhesive, there could be overlap of the adhesive portions with the heating zones without any adverse effect. Each heating zone consists of an electronic component capable of producing localized heat and a mechanism for detecting the heat within the zone. A cable, wireless or other means of electronic communication connects the bandage to a controller.

In preferred embodiments, the pixilated bandage is stored in a protective packaging unit. Storage may be individual bandages or groups of bandage elements depending on the application. The storage unit should be suitable for sterilization. In use, the user removes the bandage(s) from the protective packaging and a user peels the backing from the adhesive side of the bandage and affixes the bandage over the area to be treated. The user may also add or remove bandage elements to adjust the size and shape of the bandage for proper application The backing, which may be separate or integrated into the packaging unit, protects the adhesive layer of the bandage.

Once affixed, embodiments of the bandage establish electronic communication between the bandage and a controller unit that provides the electrical power necessary to operate the heating elements and provides the control of the heating elements to maintain a desired treatment regimen. The controller can be programmed to initialize treatment either from predetermined parameters, user selected parameters, or a combination of both. The controller exchanges electrical signals and power using whatever electrical communication path is established between the controller and the bandage. The signals and power in preferred embodiments can be used to effectuate the controlled heating of various zones within the bandage to a predetermined temperature and for a predetermined period of time. The controller can also be programmed to execute a pattern of heating that will vary the heating of different zones of the bandage over time and position within the bandage. This pattern can be selectable according to the type of treatment to be executed.

In certain embodiments, the controller will cease electrical communications with the bandage at the conclusion of the treatment regimen or program. During this conclusion of treatment certain embodiments, according the concepts described herein, allow the controller to disable the bandage by changing information stored on the bandage, disabling a fusable link, or any other mechanism which prevents the bandage from being reused. After treatment is completed the bandage can be removed. The removed bandage may be either stored for future use or disposed.

In preferred embodiments, the bandage can be constructed as a multilayer assembly. In such a multilayer assembly, the main physical layer can be formed from a flexible material. A lower layer consisting of biocompatible adhesive can be provided to affix the bandage to the skin. Distributed either under, over, or within the adhesive layer one or more heating zones are incorporated into the bandage. The configuration of the heating zones may be concentric, radial, linear array, two dimensional array or a combination of shapes. Each heating zones preferably includes a heating element and a temperature sensing device or mechanism. The heating element can be an electrically controllable heater consisting of an electronic element made from conductive, ceramic, carbon, carbon ink, metal film, resistor, inductor, transistor, or IR emitting junction. Connection between the heating elements and the electronic communication path can be accomplished by conductors made from either wire, metalized cotton, conductive ink, metallic trace or other conductive circuit trace. The electronic communication path can be any mechanism for making an electrical connector such as a cable, flexible circuit, or wireless link capable of carrying the electrical connections between the controller and the bandage. The electronic communication path can also include a protective insulating layer and an electrical or wireless connection to the controller.

Preferred embodiments allow for the heating of the bandages heating elements where the controller controls the bandage for either treatment time, treatment temperature(s) or both using electronically controlled voltage or current signals. Feedback of bandage temperature or surface temperature for control may be included to provide accuracy of treatment temperature. Temperature may be varied by heating or bandage element location, time, feedback temperature, preprogrammed temperature profile or any combination of these factors.

In preferred embodiments the time of treatment can be controlled from anywhere between around 1 second to 48 hours or longer per bandage. The treatment temperature is preferably between 38 C to 58 C.

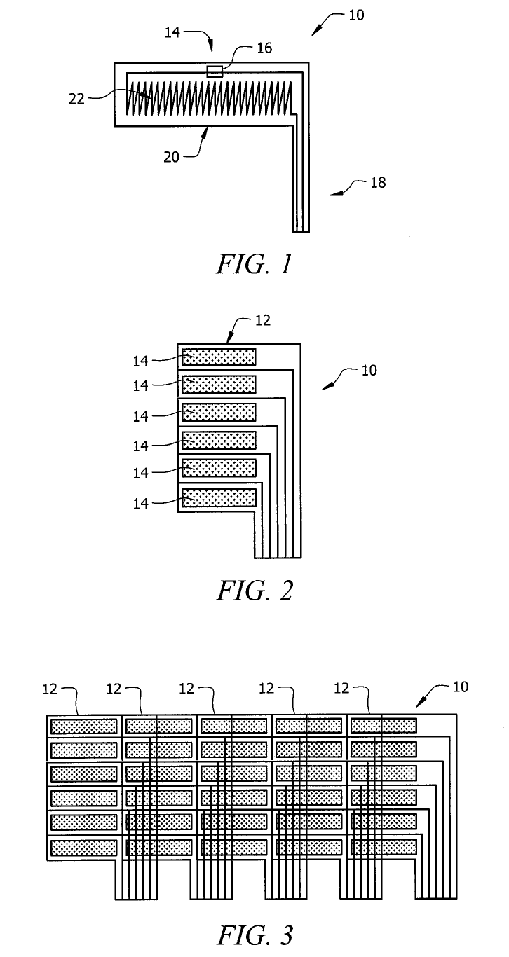

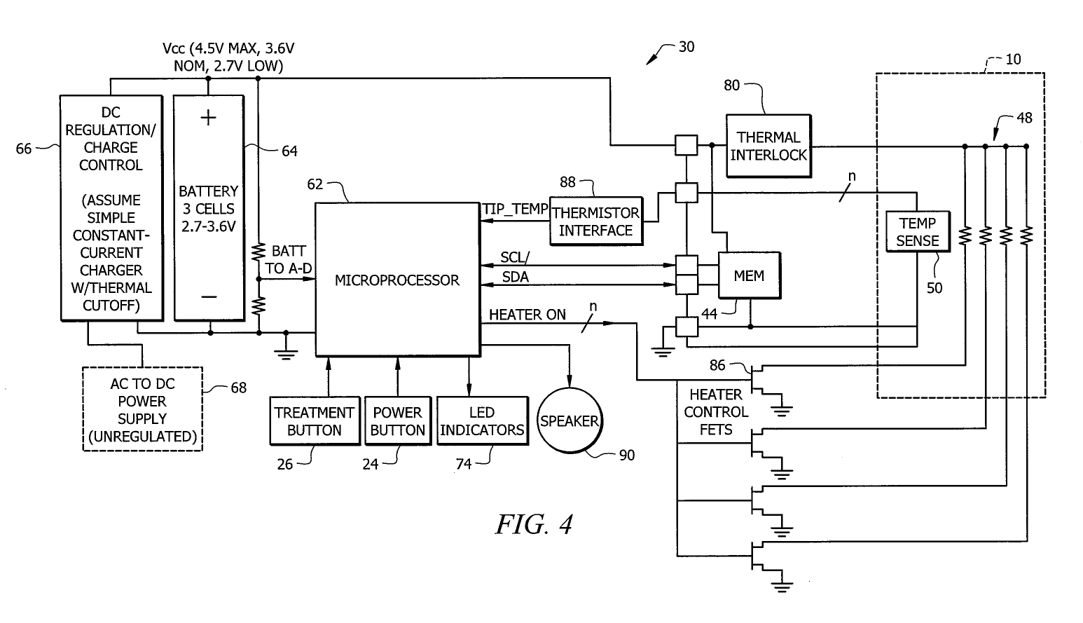

Referring now to FIGS. 1-3, an embodiment of a heated bandage structure is shown. Bandage 10 can be made up of single elements or constructed from multiple equivalent elements 14 as will be described. Each individual element 14 includes a bandage portion 20 and lead portion 18. Bandage portion 20 is intended to cover the wound or affected area and includes heating element 22. A thermistor 16, or other feedback mechanism such as a differential loop or trace may also be included to allow for the regulation of the effective treatment temperature of bandage 10. Heating element 22 is preferably a resistive heating element as has been described. Lead portion 18 provides for the electrical connections between heating element 22 and the feedback mechanism, shown here as thermistor 16 and a control unit used to control bandage 10. The controller will be described in greater detail with reference to FIGS. 4-8.

Bandage 10 may connect to the controller either by direct electrical connection or could be connected by a wireless electrical connection. In an embodiment of bandage 10, a flexible Mylar laminated circuit board can be used wherein the traces form a resistive heater and incorporate a thermal detection device localized to the heater, such as thermistor 16 or other feedback mechanism. The individual bandage can be of any size and can be very thin, such as 0.125″ tall (excluding the leads).

Referring now to FIG. 2, an embodiment of a bandage according to the present invention using a linear arrangement of heating elements is shown. Bandage 10 is formed from a linear array 12 of individual heating elements 14. Each heating element can have its own thermistor or other feedback device and each element can be individually connected to and controlled by a controller as described. By using multiple, individually controlled heating elements, bandage 10 can deliver heat only to certain areas of the area being treated or can deliver different treatment protocols to different areas covered by the bandage.

Referring now to FIG. 3, an embodiment of a bandage according to the present invention using a linear arrangement of heating elements is shown. Bandage 10 has multiple linear arrays 12 of individual heating elements arranged into a two dimensional array of individually controlled heating elements. Bandage 10 shows how multiple strips of linear pixels could be formed into an array or could be laid end to end (with leads overlapping) to form a matrix for even broader wound coverage of nearly unlimited size. With all leads connected to the same central controller, an image of the resulting matrix can be represented on a touch screen allowing a technician to turn certain areas on or off or to set varying temperatures for different areas of the wound, targeting at-risk areas with the most energy. Specific zones could be targeted or a gradient could be created either to direct heat to a certain spot or to take into account the Thermal Aspect Ratio effect in order to maintain a safe operating temperature at all times. The leads of the linear strips could be formed or arranged to overlap the adjacent strip to provide a uniform heated surface. This bandage may benefit from using a biocompatible pressure sensitive adhesive for applying to a patient

Bandages 10 from FIGS. 2 and 3 can be arranged so as to be configurable or sizable for a particular wound. In bandage 10 from FIG. 2, elements from the far end of the linear arrangement could be trimmed off without disturbing the functionality of the remaining elements. Similarly, bandage 10 from FIG. 3 could be sized by removing exterior elements of the array as long as the leads to other elements are not damaged. Trimming or removing of elements could be done by the use of scissors or the like or by preformed perforations in the bandage, or other similar mechanism.

Treatment device 10 operates to transfer heat energy to a wound, or a portion of a wound, at a set temperature for a set period of time. The set temperature and set period of time can be varied to accommodate different conditions or infections, but embodiments of treatment device 10 should be capable of heating a treatment surface to a temperature between 38° C. and 67° C. and sustaining one or more temperatures within that range for a continuous period or for a variable treatment period. Although thermal damage generally occurs when human skin is heated to a temperature of approximately 66° C. (150° F.) or greater, an interface according to an embodiment of the invention heated to this temperature or a higher temperature can nevertheless deliver an effective therapeutic amount of heat to a wound without resulting in thermal damage, depending on the amount of thermal energy delivered over a particular surface area and how readily the thermal energy is dissipated by the heated tissue.

Control of the temperature of one or more of the individual elements 14 is done in response to signals from a feedback mechanism such as thermistor 16, which provides an electrical signal indicative of the temperature of the heating element to a microprocessor in body in a controller.

Referring now to FIG. 4, a diagram showing the operation of the electrical components of an embodiment of a treatment device 30 for controlling the pixilated bandage 10 from FIGS. 1-3 is described. Device 30 includes microprocessor 62. Microprocessor 62 is programmed to respond to and control the various inputs and outputs of treatment device 30. Microprocessor 62 receives input from a power button 24, and in response operates to power-up or power-down the treatment device accordingly. Microprocessor 62 also receives input from a treatment button 26 and operates to start or stop treatment based on input from treatment button 26. LEDs 74 can be turned on and off by microprocessor 62 to communicate visual information to information to the user, while a speaker 90 can be used and controlled by microprocessor 62 to communicate audible information to the user.

Microprocessor 62 is also connected to bandage 10. Microprocessor communicates with a memory element 44 and exchanges information on programming, calibration, treatment parameters and variations and other information. Microprocessor also receives the signal from temperature feedback mechanism or mechanisms 50 through interface 88. Using the signal from temperature feedback mechanism 50, microprocessor 62 is operable to control the temperature of each of the individual elements of bandage 10. Microprocessor 62 of the illustrated embodiment is connected to the gate of field effect transistors (“FETs”) 86, and by varying the voltage at the gate of each FET 86 is able to control the amount of current flowing through resistors 48 in each element 14 of bandage 10. The heat produced by resistors 48 is proportional to the amount of current passing through them. A thermal interlock can be provided as a safety mechanism to ensure that the failure of any temperature feedback mechanism 50 does not cause a dangerous operating temperature in the bandage 10.

Microprocessor 62 can be programmed with a control algorithm referred to as a proportional, integral, derivative or PID. A PID is a control algorithm which uses three modes of operation: the proportional action is used to dampen the system response, the integral corrects for droop, and the derivative prevents overshoot and undershoot. The PID algorithm implemented in microprocessor 62 operates to bring the thermal mass to the desired operating temperature as quickly as possible with minimal overshoot, and also operates to respond to changes in the temperature of the thermal mass during the treatment cycle that are caused by the heat sink effect of the treatment area.

The controller may be configured to independently control any number of multiple heating elements where each individual element may have its own program, treatment profile or algorithm, or patterns of elements may be controlled in concert to achieve a desired treatment pattern where each element may have different treatment temperatures, treatment time or other programs as may be conceived of by one skilled in the art.

In addition to being connected to FETs 86, resistors 48 are connected to battery 64 or other power source such as an AC wall socket through thermal interlock 80, which can be a fuse having a maximum current rating chosen to prevent runaway overheating of resistors 48. Where a battery is used, the battery 64, which can be comprised of one or more individual cells, can be charged by battery charger 66 when battery charger 66 is connected to an external power supply 68. External power supply 68 can be any type of power supply, but is normally an AC to DC converter connected between battery charger 66 and an ordinary wall outlet. According to embodiments, the output voltage of battery 66 is directly related to the amount of charge left in battery 66, therefore, by monitoring the voltage across battery 66 microprocessor 62 can determine the amount of charge remaining in battery 66 and convey this information to the user using LEDs 74 or speaker 90. Other methods of determining battery voltages or charge for different battery technologies can also be used and are well within the scope of the present invention.

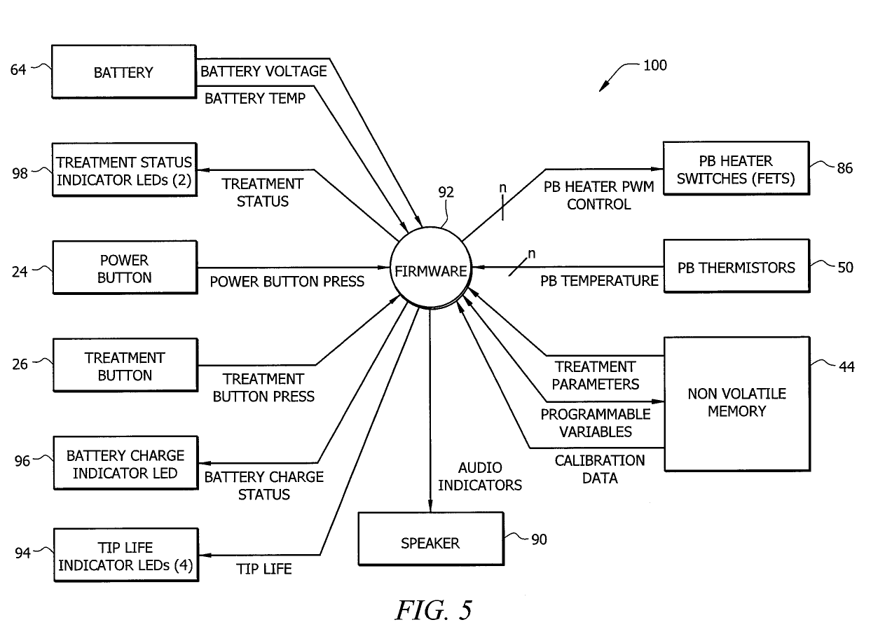

Referring now to FIG. 5, a diagram showing the various inputs to the firmware run by microprocessor 62 of FIG. 4 is described. Firmware 92 represents the programming loaded on microprocessor 62 from FIG. 4. As described with reference to FIG. 4, microprocessor 62 is operable to respond to and control the various aspects of treatment device 30 from FIG. 4. Firmware 92 is able to accept inputs from power button 70, treatment button 26, temperature feedback mechanisms 50 and battery 64. Firmware 92 is also able to exchange information with memory element 44, such as treatment parameters, programmable variables to the treatment set by a user or medical professional, calibration data, and any other information useful to the device. The microprocessor 62 and memory element 44 may exchange any other information that may increase the efficacy of treatment device 30.

In response to the temperature feedback input and information from memory element 44, firmware 92 controls FETs 86 to regulate the temperature of the individual heating elements in the bandage according to the PID algorithm programmed into firmware 92. Firmware 92 also controls speaker 90 to provide audible feedback to the user and LEDs 94, 96, and 98 which are subsets of LEDs 74 from FIG. 4, and provide indications of device and/or treatment status.

As with the control circuitry, the firmware can be configured to control multiple independent heating elements individually, in a preprogrammed pattern or in any other manner which would enhance the treatment capabilities of the device 30 or bandage 10.

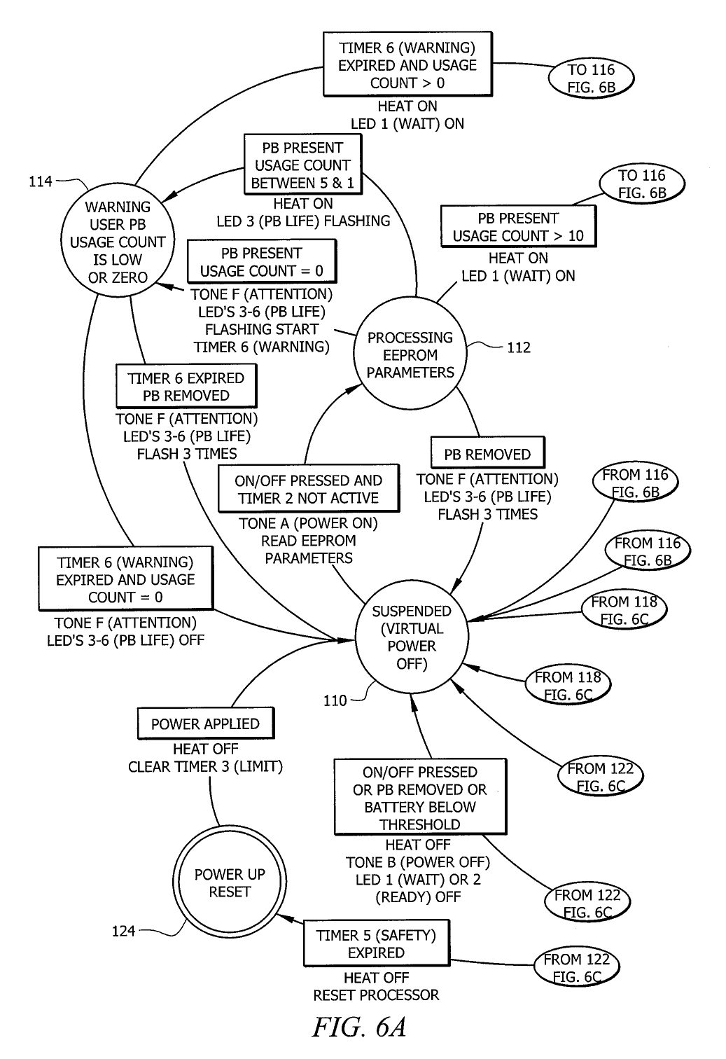

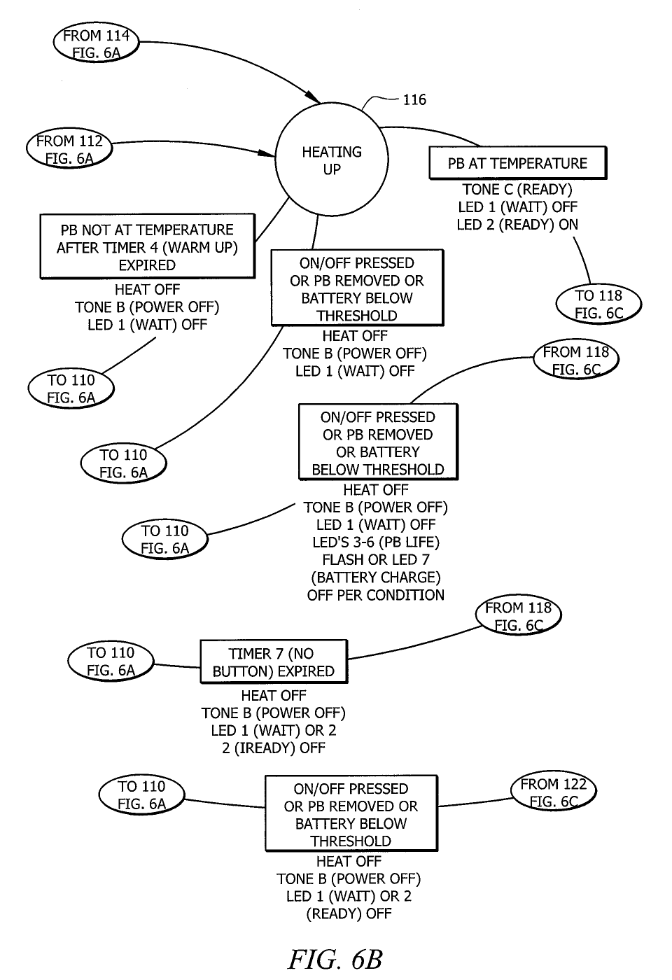

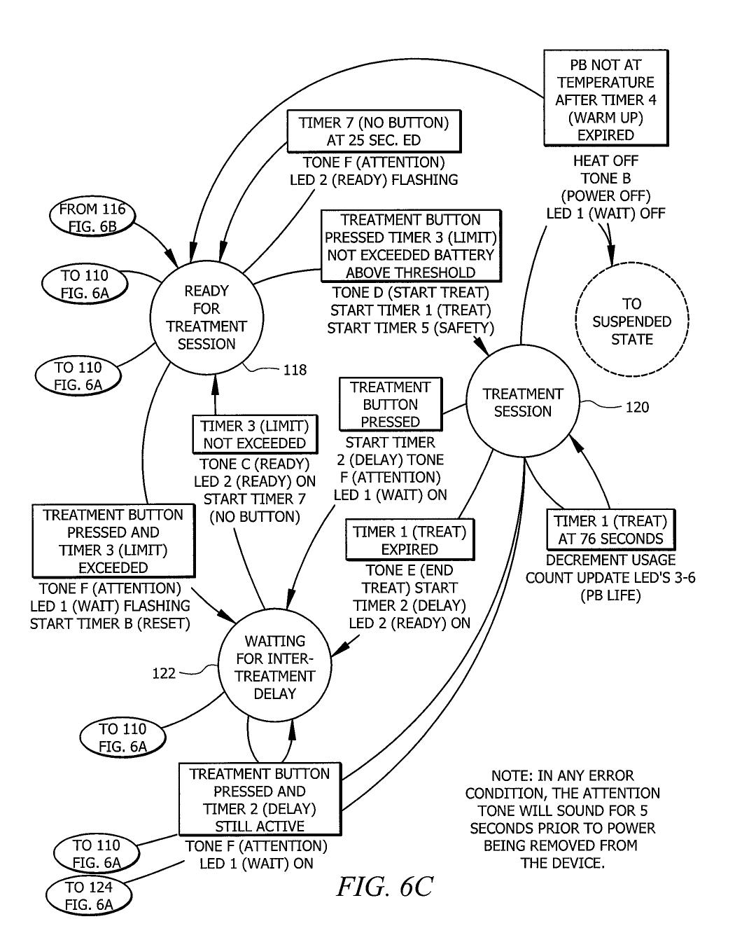

Referring now to FIGS. 6A-6C, a state transition diagram showing various operating states of firmware 92 from FIG. 5 according to an embodiment is described. The state diagram begins a Suspended state 110 which is the power off state. During the power off mode the microprocessor is still receiving some power to allow it to monitor the power button. The Suspended state 110 is left when the power on button is pressed, and the state proceeds to the Processing Memory state 112. In the Processing Memory state 112 the microprocessor 62 and memory element 44 from FIG. 4 exchange information. The state then passes to Heating state 116 (FIG. 6B). If there is an error in the exchange of information or if the treatment program is outside of predetermined safety parameters the state progresses to the Warning state 114 (FIG. 6A) which provides visual and or audible signals to the user to indicate a problem or error. If there is a problem with the memory or treatment program the state passes from the Warning state 114 to the Suspended state 110.

During the Heating state 116 the elements of the bandage are heated using resistors 48 from FIG. 4 according to the programmed treatment regimen. A predictive model is used to set a timer based on the amount of time that should be required for each element to come to temperature. This timer acts as in indicator that the heating elements are responding to the heating correctly. If one or more of the heating elements does not reach the predetermined operating temperature by the expiration of the timer, it is an indication of a potentially faulty component and the treatment device can be shut down by transitioning to Suspended state 110. or a warning of a faulty element can be provided using signaling by LED or speaker, while proceeding with the treatment. Other predictions of heating element behavior can also be used to detect potentially faulty components.

In addition to the expiration of the timer, the treatment device powers down by transitioning to the Suspended state if the power button is pressed, the bandage is removed or the power source voltage falls below a threshold, and indication of the fault is provided to the user through visual and/or audible signals. If the heating elements successfully reach the operating temperature within the designated time the state transitions to Ready state 118. A timer is started upon entering the Ready state 118. If the timer expires or the power button is pressed while in the Ready state 118, the state transitions to the Suspended state 110.

The device may proceed directly to Treatment state 120 or may be programmed to require an addition input such as the pressing of the power button while in the Ready state 118 before transitioning to Treatment state 120. In certain embodiments two timers, a treatment timer and a safety timer, can be used, while in other embodiments only one timer may be used. Each timer that is used is started upon entering the Treatment state 120. The safety timer is slightly longer than the treatment timer so that if there is a failure in the treatment timer the safety timer will expire and transition the state to the Power Reset state 124 before transitioning to the Suspended state 110. The state also transitions from Treatment state 120 to Suspended state 110 if the power button is pressed during a treatment cycle.

As a treatment cycle can be a relatively long period of time, the treatment device can also be programmed to provide visual and/or audible indications of the progress of the treatment timer. For example, speaker 90 of FIG. 4 can be used to provide intermittent tones during the treatment to let the user know that the treatment is continuing. The time between the tones could be spaced to provide an indication of the remaining time in the treatment cycle, such as by shortening the time between the tones as the cycle gets closer to the end. Many other methods of providing visual or audible feedback could be contemplated and are well within the scope of the present invention.

When the treatment timer expires, or if there is operator input, the state transitions from Treatment state 120 to Wait state 122 which forces an inter-treatment delay. If there is additional operator input or the bandage is removed during the Wait state, the state transitions to Suspended state 110. After the expiration of the inter-treatment delay the state transitions back to Ready state 118. In addition to the inter-treatment delay, the Wait state 122 can be used to force a temporal treatment limit. While the inter-treatment delay forces a relatively brief delay between treatment cycles, the temporal treatment limit can be used in certain treatment situations to limit the number of treatments that can be performed in specified period. For example, if the treatment cycle is two and a half minutes and the inter-treatment delay is 10 seconds, a temporal treatment limit of 30 minutes could be used to limit the device to approximately 10 to 11 consecutive treatments before a forced interval is imposed.

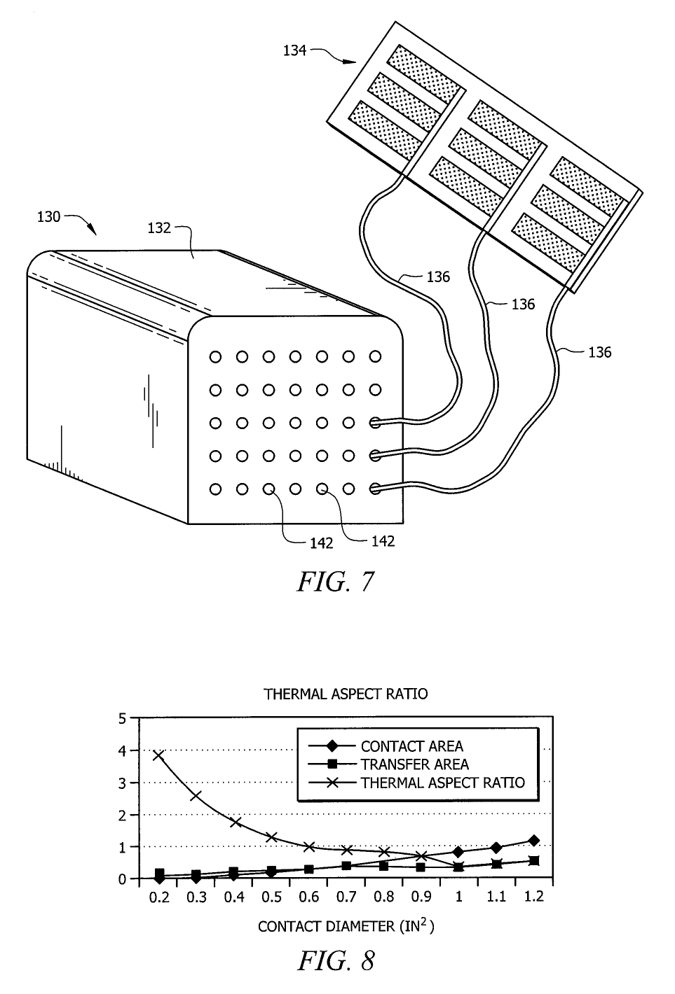

Referring now to FIG. 7, an embodiment of a physical treatment device 130 is shown. Device 130 includes treatment base station 132 and pixilated bandage 134. Base station 132 includes all the essential functionality described in FIGS. 4-6. The device includes multiple connections to control one or more individual elements of a bandage 10 from FIGS. 1-3. While bandage 134 is shown with a particular number of elements, device 130 can be constructed to control any number of individual elements arranged in any manner. Further, while the device 130 is shown as having one connection per linear array in bandage 134, any connection mechanism can be used and the elements can be controlled individually, as subgroups or as a whole bandage while remaining within the scope of the concepts described herein.

Base station 132 communicates with bandage 134 either by wires 136 connecting the base station 132 to bandage 134, as is shown in FIG. 7, or by means of a wireless connection. The base station 132 includes LEDs and a speaker as described above to convey visual and audio information to the user of device 130. Base station 130 may also include a connection to allow base station 130 to be connected to a computer where the computer is programmed to have an interface to control the treatment parameters and to allow control over individual elements in a user friendly fashion.

The aspect ratio between a thermal transfer area and thermal contact area plays a significant role in determining the internal skin temperature resulting from a given size of bandage. This Thermal Aspect Ratio should be used to design appropriate treatment devices, as well as to drive predictive models on given design specifications.

The treatment device of the present invention relies on a thermal contact area used to heat a limited region of the skin to a temperature sufficient to induce heat shock in bacterial, viral or fungal infections. The size and temperature of the elements in the bandage are tuned to result in a carefully targeted temperature which is sufficient to induce heat shock, but not high enough to create significant or permanent skin damage.

The existing research on heat transfer and contact burns focuses on a fixed (and relatively large) contact area (typically 7 cm² or larger). These research studies attempt to create predictive models of burn incidence at varying temperatures and times. Reducing the size of the contact area, however, can produce a dramatic reduction in burn incidence. This reduction does not occur in a linear relationship. This non-linear relationship is largely the result of the Thermal Aspect Ratio shown in FIG. 8, which is also non-linear as a result of the inherent geometry of the two components (contact area and transfer area). As the diameter of the contact area increases, the ratio of the transfer area to the contact area drops off dramatically at first and then much more gradually as the contact diameter goes above 0.60 inches.

Since the contact area increases with the square of the contact radius and the transfer area is essentially a fixed width band around the circumference of the contact area, it follows that drop in the Thermal Aspect Ratio is initially steep.

This analysis relies on a fixed heat transfer coefficient for skin. The presumption is that this fixed coefficient results in a fixed transfer area width (0.125″) which is the area immediately surrounding the circumference of the contact area through which the higher temperature of the contact area is wicked away and dissipated through contact with the air and blood-circulating skin mass. Because of the fixed heat transfer coefficient, the transfer area acts much like a fence, preventing additional heat transfer beyond that which is permitted by its own heat transfer coefficient.

When the Thermal Aspect Ratio is high, the contact area gets relatively good and uniform heat dissipation via the transfer area (in addition to the heat transfer via blood flow and body mass contact directly beneath the contact area). As the Thermal Aspect Ratio drops, a larger and larger contact area takes on more and more heat energy which cannot be dissipated via the transfer area resulting in rapid heat build-up. In essence, larger contact areas lose their ability to shed heat and ramp up to higher temperatures at a rapidly increasing rate.

The Thermal Aspect Ratio dynamic, therefore, creates an inflection point in contact area. Before the inflection point (areas below a certain point), a relatively high capacity for dissipation allows the use of higher temperature therapy to a concentrated area without significant risk of thermal damage. Beyond the inflection point (contact areas above a certain point) maintaining a safe and predictable temperature becomes more and more difficult to do and operating temperature (and therefore, therapy temperature) must come down in order to avoid thermal damage.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A bandage comprising:
a plurality of individual heating elements, at least a subset of elements of the plurality of individual elements being able to be separately controlled independent of the other elements to direct thermal energy to a specific area of the bandage; and
a controller operable to control the plurality of heating elements forming the bandage.

2. The bandage of claim 1 wherein the individual heating elements are arranged in a linear array.

3. The bandage of claim 1 wherein the individual heating elements are arranged in two dimensional array.

4. The bandage of claim 1 wherein the controller is operable to maintain the individual heating elements within a defined temperature range.

5. The bandage of claim 1 wherein the controller is operable to control the individual heating elements according to a preprogrammed treatment regimen.

6. The bandage of claim 1 wherein the thermal energy is sufficient to kill bacteria.

7. The bandage of claim 1 wherein each heating element is formed on a flexible circuit board.

8. The bandage of claim 7 wherein the circuit board is formed of mylar.

9. The bandage of claim 1 wherein each individual heating element includes a feedback mechanism allowing the controller to react to temperature changes.

10. A method of delivering thermal energy to a wound comprising:
placing a bandage on the wound the bandage formed of a plurality of independently controllable elements, each element including a heating element and a feedback mechanism;
heating one or more of the plurality of independently controllable elements to a temperature sufficient to destroy infectious agents; and
controlling the one or more of the plurality of independently controllable heating elements using the feedback mechanism to deliver a therapeutic amount of thermal energy according to a preprogrammed treatment regimen.

11. The method of claim 10 wherein the plurality of independently controllable elements are formed in a two dimensional array.

12. The method of claim 11 wherein the bandage can be modified to fit a particular wound.

13. The method of claim 10 further comprising maintaining the temperature of the heating elements within a predetermined safe operating range of temperatures.

14. The method of claim 10 wherein the treatment regimen includes maintaining selected elements of the plurality of independently controllable elements at a temperature for a set time

15. The method of claim 14 wherein the treatment regimen includes multiple treatment cycles.

16. A bandage element comprising:
a heating element formed on a flexible circuit board:
a feedback mechanism formed on the flexible circuit board operable to provide a signal indicative of the temperature of the heating element; and
leads extending from the heating element and feedback mechanism providing electrical connection of the heating element and the feedback mechanism to a controller;
wherein the bandage element is configurable with other bandage elements into an array of individually controllable bandage elements.

17. The bandage element of claim 16 wherein the feedback mechanism is a thermistor.

18. The bandage element of claim 16 wherein the flexible circuit board is formed of mylar.

19. The bandage element of claim 16 wherein the array is a linear array.

20. The bandage element of claim 16 wherein the array is a two dimensional array.

21. The bandage element of claim 16 wherein the controller is operable to maintain the heating element within a particular temperature range for a predetermined amount of time.

22. The bandage element of claim 16 wherein the heating element is capable of being heated to a temperature sufficient to destroy infectious agents.
